(19) United States
(12) Patent Application Publication
(10) Pub. No.: US 2013/0289080 A1
(43) Pub. Date: Oct. 31, 2013

(54) COSMETIC COMPOSITION COMPRISING A FIXING POLYMER AND AN ANTIDANDRUFF AGENT

(71) Applicants: Virginie Masse, Clichy (FR); Christine Annotel, Paris (FR); Cécile Bebot, Clichy (FR)

(72) Inventors: Virginie Masse, Clichy (FR); Christine Annotel, Paris (FR); Cécile Bebot, Clichy (FR)

(73) Assignee: L'OREAL, Paris (FR)

(21) Appl. No.: 13/821,672

(22) PCT Filed: Sep. 14, 2011
(86) PCT No.: PCT/EP2011/065918

§ 371 Date: Mar. 8, 2013

Related U.S. Application Data
(60) Provisional application No. 61/385,225, filed on Sep. 22, 2010.

(30) Foreign Application Priority Data
Sep. 14, 2010 (FR) 1057311

(51) Int. Cl. A61K 8/49; A61Q 5/00

(52) U.S. Cl. CPC A61K 8/4926; A61Q 5/006
USPC 514/345

(57) ABSTRACT

The invention relates to a cosmetic composition comprising, in a cosmetically acceptable medium: one or more fixing polymer(s) (i), one or more associative polymer(s) (ii) different from the fixing polymer(s), one or more antidandruff agent(s) (iii), and to a process using this composition, and to the use thereof.

The present patent application relates to a cosmetic composition and to a process for treating keratin materials, preferably human keratin materials and in particular the scalp and the hair.

Dandruff problems affect up to 50% of the worldwide population. They affect both men and women and are perceived as having a very negative psychosocial impact. The appearance of dandruff is disagreeable both aesthetically and because of the irritation it causes (itching, redness, etc.), and as such many people confronted with this problem to variable degrees wish to eliminate it efficiently and permanently.

Dandruff corresponds to excessive and visible desquamation of the scalp resulting from excessively rapid multiplication of the epidermal cells and their abnormal maturation. This phenomenon may be caused especially by microtraumas of physical or chemical nature, such as excessively aggressive hair treatments, extreme climatic conditions, nervousness, the diet, fatigue and pollution, but it has been demonstrated that dandruff conditions usually result from a disorder of the microflora of the scalp and are more particularly due to the excessive colonization of a fungus belonging to the family of yeasts of the genus Malassezia (previously known as Pityrosporum) and which is naturally present on the scalp.

To treat dandruff, it is known practice to use antidandruff agents, especially antifungal and/or antibacterial agents in a medium capable of distributing these agents and depositing them onto the teguments.

These antidandruff agents are usually formulated in fluid lotions.

There is a need felt by users for antidandruff treatments in the form of thickened and preferably gelled compositions, for enabling better localization while at the same time conserving good antidandruff efficacy. The difficulty is thus that of obtaining products with a clear medium with the absence of residues on the head arising from the medium.

In addition, antidandruff compositions that are stable and that have good working properties, especially during the application or removal of the compositions, are sought.

The Applicant has discovered, surprisingly and advantageously, that the use of a combination of at least one fixing polymer and of at least one antidandruff agent and at least one associative polymer makes it possible to obtain thickened and in particular gelled, clear antidandruff compositions that do not leave residues on the head arising from the formulation medium.

In addition, these antidandruff compositions are stable and have good working properties, especially during the application of the compositions. They can also give the head of hair advantageous hold properties.

One subject of the invention is a cosmetic composition comprising, in a cosmetically acceptable medium:
one or more fixing polymer(s) (i),
one or more associative polymer(s) (ii) different from the fixing polymer(s),
one or more antidandruff agent(s) (iii).

Other characteristics, aspects, objects and advantages of the present invention will emerge even more clearly on reading the description and the examples that follow.

The cosmetic composition according to the invention may be in the form of a spray, a mousse or a gel and is preferably in the form of a gel.

Another subject of the present invention consists of a cosmetic treatment process in which the cosmetic composition according to the invention is used on keratin materials, preferably human keratin materials and in particular the scalp and the hair.

A third subject of the invention concerns the uses of this cosmetic composition for the antidandruff treatment of the hair and the scalp.

Fixing Polymer

The expression “fixing polymer” is understood within the meaning of the present invention to mean any polymer that makes it possible to give a shape to the hair or to hold the hair in a given shape.

All the nonionic, anionic, cationic or amphoteric fixing polymers and mixtures thereof used in the field of haircare may be used in the compositions according to the present patent application.

The fixing polymers may be soluble in the cosmetically acceptable medium or insoluble in this same medium and used in this case in the form of dispersions of solid or liquid particles of polymer (latex or pseudolatex).

The fixing polymers of the invention preferably do not contain any C₈-C₃₀ fatty chains.

Anionic polymers that may be mentioned include polymers comprising groups derived from carboxylic acids, sulfonic acids or phosphoric acids, and having a weight-average molecular mass of between 500 and 5 000 000.

The carboxylic groups are provided by unsaturated monocarboxylic or dicarboxylic acid monomers such as those corresponding to the formula:

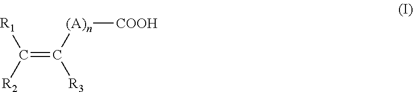

in which n is an integer from 0 to 10, A denotes a methylene group, optionally connected to the carbon atom of the unsaturated group or to the neighbouring methylene group when n is greater than 1 via a heteroatom such as oxygen or sulfur, R₁ denotes a hydrogen atom or a phenyl or benzyl group, R₂ denotes a hydrogen atom or a lower alkyl or carboxyl group, and R₃ denotes a hydrogen atom, a lower alkyl group or a —CH₂—COOH, phenyl or benzyl group.

In formula (I) above, a lower alkyl group preferably contains from 1 to 4 carbon atoms and in particular denotes methyl and ethyl groups.

The anionic fixing polymers containing carboxylic or sulfonic groups that are preferred are:

A) copolymers of acrylic or methacrylic acid or salts thereof, including copolymers of acrylic acid and of acrylamide and methacrylic acid/acrylic acid/ethyl acrylate/methyl methacrylate copolymers, in particular Amerhold DR 25 sold by the company Amerchol. Mention may also be made of methacrylic acid/ethyl acrylate copolymers, especially as an aqueous dispersion, such as Luviflex Soft and Luvimer MAE sold by the company BASF. Mention may also be made of butyl acrylate/acrylic acid/methacrylic acid branched block copolymers such as Fixate G100L sold by the company Lubrizol.

B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol and optionally crosslinked. Such polymers are described in particular in French patent 1 222 944 and German patent application 2 330 956, the copolymers of this type comprising an optionally N-alkylated and/or hydroxyalkylated acrylamide unit in their chain as described especially in Luxembourg patent applications 75370 and 75371. Mention may also be made of copolymers of acrylic acid and of C₁-C₄ alkyl methacrylate.

C) crotonic acid-based copolymers, such as those comprising in their chain vinyl acetate or propionate units and optionally other monomers such as allylic or methallylic esters, vinyl ether or vinyl ester of a saturated, linear or branched carboxylic acid containing a hydrocarbon-based chain, these polymers possibly being grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products that fall within this category are the resins 28-29-30, 26-13-14 and 28-13-10 sold by the company
National Starch.

Crotonic acid-based copolymers that may also be mentioned include crotonic acid/vinyl acetate/vinyl t-butylbenzoate terpolymers and in particular Mexomer PW supplied by the company Chimex.

D) polymers derived from maleic, fumaric or itaconic acids or anhydrides with vinyl esters, vinyl ethers, vinyl halides, phenylvinyl derivatives or acrylic acid and esters thereof; these polymers may be esterified. Such polymers are described in particular in U.S. Pat. Nos. 2,047,398, 2,723,248 and 2,102,113 and GB patent 839 805, and especially those sold under the names Gantrez® AN or ES by the company ISP.

Polymers also falling within this category are the copolymers of maleic, citraconic or itaconic anhydrides and of an allylic or methallylic ester optionally comprising an acrylamide or methacrylamide group, an α-olefin, acrylic or methacrylic esters, acrylic or methacrylic acids or vinylpyrrolidone in their chain, the anhydride functions being monoesterified or monoamidated. These polymers are described, for example, in French patents 2 350 384 and 2 357 241 by the Applicant.

E) polyacrylamides comprising carboxylate groups.

F) polymers comprising sulfonic groups. These polymers may be polymers comprising vinylsulfonic, styrenesulfonic, naphthalenesulfonic, acrylamidoalkylsulfonic or sulfoisophthalate units.

These polymers can be chosen in particular from:
polyvinylsulfonic acid salts with a molecular mass of between about 1000 and 100 000, and also copolymers with an unsaturated comonomer such as acrylic or methacrylic acids and esters thereof, and also acrylamide or derivatives thereof, vinyl ethers and vinylpyrrolidone;
polystyrenesulfonic acid salts, sodium salts, with a molecular mass of about 500 000 and of about 100 000. These compounds are described in patent FR 2 198 719;
polyacrylamidesulfonic acid salts such as those mentioned in U.S. Pat. No. 4,128,631.

G) grafted anionic silicone polymers.

The grafted silicone polymers used are preferably chosen from polymers containing a non-silicone organic backbone grafted with monomers containing a polysiloxane, polymers containing a polysiloxane backbone grafted with non-silicone organic monomers, and mixtures thereof.

In the text hereinabove or hereinbelow, in accordance with what is generally accepted, the terms “silicone” and “polysiloxane” are understood to denote any organosilicon polymer or oligomer having a linear or cyclic, branched or crosslinked structure, of variable molecular weight, obtained by polymerization and/or polycondensation of suitably functionalized silanes, and consisting essentially of a repetition of main units in which the silicon atoms are linked together by oxygen atoms (siloxane bonding ≡Si—O—Si≡), optionally substituted hydrocarbon-based radicals being linked directly via a carbon atom to the said silicon atoms. The most common hydrocarbon-based radicals are alkyl radicals, especially C₁-C₁₀ alkyl radicals, and in particular methyl, fluoroalkyl radicals, aryl radicals and in particular phenyl, and alkenyl radicals and in particular vinyl; other types of radicals which can be linked, either directly or via a hydrocarbon-based radical, to the siloxane chain are, especially, hydrogen, halogens and in particular chlorine, bromine or fluorine, thiols, alkoxy radicals, polyoxyalkylene (or polyether) radicals and in particular polyoxyethylene and/or polyoxypropylene radicals, hydroxyl or hydroxyalkyl radicals, substituted or unsubstituted amine groups, amide groups, acyloxy or acyloxyalkyl radicals, hydroxyalkylamino or aminoalkyl radicals, quaternary ammonium groups, amphoteric or betaine groups, anionic groups such as carboxylates, thioglycolates, sulfosuccinates, thiosulfates, phosphates and sulfates, needless to say this list not being limiting in any way (“organomodified” silicones).

In the text hereinbelow, in accordance with what is generally accepted, the term “polysiloxane macromer” means any monomer containing in its structure a polymer chain of the polysiloxane type.

The polymers containing a non-silicone organic backbone grafted with monomers containing a polysiloxane, used according to the present invention, consist of an organic main chain formed from organic monomers not comprising silicone, onto which is grafted, within the said chain and also optionally on at least one of its ends, at least one polysiloxane macromer.

The non-silicone organic monomers constituting the main chain of the grafted silicone polymer can be chosen from free-radical-polymerizable monomers containing ethylenic unsaturation, polycondensation-polymerizable monomers, such as those forming polyamides, polyesters or polyurethanes, and ring-opening monomers, such as those of the oxazoline or caprolactone type.

The polymers containing a non-silicone organic backbone grafted with monomers containing a polysiloxane that may be used can be obtained according to any means known to those skilled in the art, in particular by reaction between (i) a starting polysiloxane macromer which is correctly functionalized on the polysiloxane chain and (ii) one or more non-silicone organic compounds, themselves correctly functionalized with a function which is capable of reacting with the functional group(s) borne by the said silicone, forming a covalent bond; a classic example of such a reaction is the free-radical reaction between a vinyl group borne on one of the ends of the silicone with a double bond of a monomer containing ethylenic unsaturation in the main chain.

The polymers with a non-silicone organic backbone grafted with monomers containing a polysiloxane, in accordance with the invention, are chosen more preferentially from those described in U.S. Pat. No. 4,693,935, U.S. Pat. No. 4,728,571 and U.S. Pat. No. 4,972,037 and patent applications EP-A-0 412 704, EP-A-0 412 707, EP-A-0 640 105 and WO 95/00578. These are copolymers obtained by radical polymerization from ethylenically unsaturated monomers and from silicone macromers containing a vinyl end group, or alternatively copolymers obtained by reaction of a polyolefin comprising functionalized groups and of a polysiloxane macromer containing an end function that is reactive with the said functionalized groups.

One particular family of grafted silicone polymers which is suitable for carrying out the present invention consists of grafted silicone copolymers comprising:

a) from 0 to 98% by weight of at least one free-radical-polymerizable lipophilic monomer (A) of low polarity containing ethylenic unsaturation;

b) from 1% to 98% by weight of at least one polar hydrophilic monomer (B) containing ethylenic unsaturation, which is copolymerizable with the monomer(s) of the type (A);

c) from 0.01% to 50% by weight of at least one polysiloxane macromer (C) of general formula:

X(Y)ₙSi(R)₃₋ₘZₘ (II)

in which:

X denotes a vinyl group which is copolymerizable with the monomers (A) and (B);

Y denotes a divalent bonding group;

R denotes a hydrogen, a C₁-C₆ alkyl or alkoxy, or a C₆-C₁₂ aryl;

Z denotes a monovalent polysiloxane unit with a number-average molecular weight of at least 500;

n is 0 or 1 and m is an integer ranging from 1 to 3; the percentages being calculated relative to the total weight of the monomers (A), (B) and (C).

These polymers, and processes for preparing them, are described in U.S. Pat. No. 4,963,935, U.S. Pat. No. 4,728,571 and U.S. Pat. No. 4,972,037 and patent applications EP-A-0 412 704, EP-A-0 412 707 and EP-A-0 640 105. They have a number-average molecular weight ranging from 10 000 to 2 000 000 and preferably a glass transition temperature Tg or a crystal melting temperature Tm of at least −20° C.

As examples of lipophilic monomers (A), mention may be made of acrylic or methacrylic acid esters of C₁-C₁₈ alcohols; styrene; polystyrene macromers; vinyl acetate; vinyl propionate; α-methylstyrene; tert-butylstyrene; butadiene; cyclohexadiene; ethylene; propylene; vinyltoluene; acrylic or methacrylic acid esters of a 1,1-dihydroperfluoroalkanol or of homologues thereof acrylic or methacrylic acid esters of an ω-hydridofluoroalkanol; acrylic or methacrylic acid esters of a fluoroalkylsulfoamido alcohol; acrylic or methacrylic acid esters of a fluoroalkyl alcohol; acrylic or methacrylic acid esters of a fluoroether alcohol; or mixtures thereof.

The preferred monomers (A) are chosen from the group consisting of n-butyl methacrylate, isobutyl methacrylate, tert-butyl acrylate, tert-butyl methacrylate, 2-ethylhexyl methacrylate, methyl methacrylate, 2-(N-methylperfluorooctanesulfoamido)ethyl acrylate and 2-(N-butylperfluorooctanesulfoamido)ethyl acrylate, and mixtures thereof.

As examples of polar monomers (B), mention may be made of acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl(meth)acrylates, diallyldimethylammonium chloride, vinylpyrrolidone, vinyl ethers, maleimides, vinylpyridine, vinylimidazole, heterocyclic vinyl polar compounds, styrene sulfonate, allyl alcohol, vinyl alcohol and vinylcaprolactam, or mixtures thereof. The preferred monomers (B) are chosen from the group consisting of acrylic acid, N,N-dimethylacrylamide, dimethylaminoethyl methacrylate, quaternized dimethylaminoethyl methacrylate and vinylpyrrolidone, and mixtures thereof.

As polar monomers (B), the anionic grafted silicone polymers used according to the invention contain at least one anionic monomer.

The preferred polysiloxane macromers (C) of formula (I) are chosen from those corresponding to the general formula (III) below:

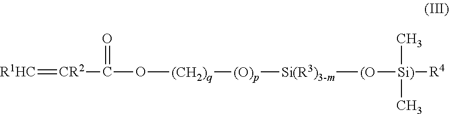

in which:

R¹ is hydrogen or —COOH (preferably hydrogen);

R² is hydrogen, methyl or —CH₂COOH (preferably methyl);

R³ is a C₁-C₆ alkyl, alkoxy or alkylamino, aryl or hydroxyl group (preferably methyl);

R⁴ is a C₁-C₆ alkyl, alkoxy or alkylamino, aryl or hydroxyl group (preferably methyl);

q is an integer from 2 to 6 (preferably 3);

p is 0 or 1;

r is an integer from 5 to 700;

m is an integer from 1 to 3 (preferably 1).

Use is made more particularly of the polysiloxane macromers of formula:

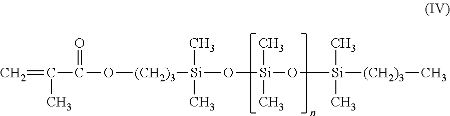

n being an integer ranging from 5 to 700.

One particular embodiment of the invention consists in using a copolymer that may be obtained by free-radical polymerization starting with the monomer mixture consisting of:

a) 60% by weight of tert-butyl acrylate;

b) 20% by weight of acrylic acid;

c) 20% by weight of silicone macromer of formula:

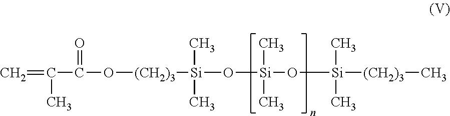

n being an integer ranging from 5 to 700; the weight percentages being calculated relative to the total weight of the monomers.

Another particular family of silicone polymers that is suitable for carrying out the present invention consists of grafted silicone copolymers which may be obtained by reactive extrusion-moulding of a polysiloxane macromer with a reactive terminal function on a polymer of the polyolefin type comprising reactive groups capable of reacting with the terminal function of the polysiloxane macromer to form a covalent bond for grafting the silicone onto the main chain of the polyolefin.

These polymers are described, along with a process for their preparation, in patent application WO 95/00578.

The reactive polyolefins are preferably chosen from polyethylenes and polymers of ethylene-derived monomers such as propylene, styrene, alkylstyrenes, butylene, butadiene, (meth)acrylates, vinyl esters or equivalents, comprising reactive functions capable of reacting with the terminal function of the polysiloxane macromer. They are chosen more particularly from copolymers of ethylene or of ethylene derivatives and of monomers chosen from those comprising a carboxylic function such as (meth)acrylic acid; those comprising an acid anhydride function such as maleic anhydride; those comprising an acid chloride function such as (meth)acryloyl chloride; those comprising an ester function such as (meth)acrylic acid esters; those comprising an isocyanate function.

The silicone macromers are preferably chosen from polysiloxanes comprising a functionalized group, at the end of the polysiloxane chain or close to the end of the said chain, chosen from the group consisting of alcohols, thiols, epoxy groups and primary and secondary amines, and more particularly from those corresponding to the general formula (VI):

T-(CH₂)ₛ—Si—[(OSiR⁵R⁶)ₜ—R⁷]_{y} (VI)

in which T is chosen from the group formed by NH₂, NHR′, an epoxy function, OH, SH; R⁵, R⁶, R⁷ and R′, independently, denote a C₁-C₆ alkyl, phenyl, benzyl or alkylphenyl, hydrogen; s is a number from 2 to 100; t is a number from 0 to 1000 and y is a number from 1 to 3. They have a number-average molecular weight preferably ranging from 5000 to 300 000, more preferentially from 8000 to 200 000 and more particularly from 9000 to 40 000.

According to the present invention, the grafted silicone polymer(s) with a polysiloxane backbone grafted with non-silicone organic monomers comprise a silicone (or polysiloxane (≡Si—O—)ₙ) main chain onto which is grafted, within the said chain and also optionally on at least one of its ends, at least one organic group not comprising silicone.

The polymers with a polysiloxane backbone grafted with non-silicone organic monomers, according to the invention, can be existing commercial products or alternatively can be obtained according to any means known to those skilled in the art, in particular by reaction between (i) a starting silicone which is correctly functionalized on one or more of these silicon atoms, and (ii) a non-silicone organic compound which is itself correctly functionalized with a function which is capable of reacting with the functional group(s) borne by the said silicone, forming a covalent bond; a classic example of such a reaction is the hydrosilylation reaction between ≡Si—H groups and vinyl groups CH₂═CH—, or alternatively the reaction between thio-functional groups —SH with these same vinyl groups.

Examples of polymers with a polysiloxane backbone grafted with non-silicone organic monomers that are suitable for carrying out the present invention, and also their specific mode of preparation, are described in particular in patent applications EP-A-0 582 152, WO 93/23009 and WO 95/03776, the teachings of which are included in their entirety in the present description by way of non-limiting references.

According to a particularly preferred embodiment of the present invention, the silicone polymer with a polysiloxane backbone grafted with non-silicone organic monomers which is used comprises the result of the free-radical copolymerization between, on the one hand, at least one non-silicone anionic organic monomer containing ethylenic unsaturation and/or a non-silicone hydrophobic organic monomer containing ethylenic unsaturation, and, on the other hand, a silicone containing in its chain at least one functional group capable of reacting with the said ethylenic unsaturations of the said non-silicone monomers, forming a covalent bond, in particular thio-functional groups.

According to the present invention, the said anionic monomers containing ethylenic unsaturation are preferably chosen, alone or as mixtures, from linear or branched, unsaturated carboxylic acids, optionally partially or totally neutralized in the form of a salt, it being possible for this or these unsaturated carboxylic acids to be, more particularly, acrylic acid, methacrylic acid, maleic acid, maleic anhydride, itaconic acid, fumaric acid and crotonic acid. The suitable salts are, in particular, alkali metal salts, alkaline-earth metal salts and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

According to the present invention, the hydrophobic monomers containing ethylenic unsaturation are preferably chosen, alone or as mixtures, from acrylic acid esters of alkanols and/or methacrylic acid esters of alkanols. The alkanols are preferably C₁-C₁₈ and more particularly C₁-C₁₂. The preferred monomers are chosen from the group consisting of isooctyl(meth)acrylate, isononyl(meth)acrylate, 2-ethylhexyl(meth)acrylate, lauryl(meth)acrylate, isopentyl(meth)acrylate, n-butyl(meth)acrylate, isobutyl(meth)acrylate, methyl(meth)acrylate, tert-butyl(meth)acrylate, tridecyl(meth)acrylate and stearyl(meth)acrylate, or mixtures thereof.

One family of silicone polymers containing a polysiloxane backbone grafted with non-silicone organic monomers that is particularly suitable for carrying out the present invention consists of silicone polymers comprising in their structure the unit of formula (VII) below:

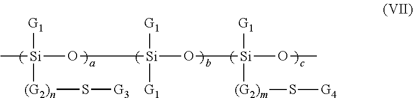

in which the radicals G₁, which may be identical or different, represent hydrogen, a C₁-C₁₀ alkyl radical or a phenyl radical; the radicals G₂, which may be identical or different, represent a C₁-C₆ alkyl group; G₃ represents a polymer residue resulting from the (homo)polymerization of at least one anionic monomer containing ethylenic unsaturation; G₄ represents a polymer residue resulting from the (homo)polymerization of at least one hydrophobic monomer containing ethylenic unsaturation; m and n are equal to 0 or 1; a is an integer ranging from 0 to 50; b is an integer which may be between 10 and 350, c is an integer ranging from 0 to 50; with the proviso that one of the parameters a and c is other than 0.

Preferably, the unit of formula (VII) above has at least one, and even more preferably all, of the following characteristics:
the radicals G₁ denote an alkyl radical, preferably a methyl radical;
n is non-zero, and the radicals G₂ represent a divalent C₁-C₃ radical, preferably a propylene radical;
G₃ represents a polymer radical resulting from the (homo)polymerization of at least one monomer of the carboxylic acid type containing ethylenic unsaturation, preferably acrylic acid and/or methacrylic acid;
G₄ represents a polymer radical resulting from the (homo)polymerization of at least one monomer of the (C₁-C₆)alkyl(meth)acrylate type, preferably isobutyl or methyl(meth)acrylate.

Examples of grafted silicone polymers corresponding to formula (IV) are, in particular, polydimethylsiloxanes (PDMSs) onto which are grafted, via a thiopropylene-type connecting chain, mixed polymer units of the poly(meth)acrylic acid type and of the polymethyl(meth)acrylate type.

Preferably, the number-average molecular mass of the silicone polymers containing a polysiloxane backbone grafted with non-silicone organic monomers of the invention ranges from 10 000 to 1 000 000 approximately and even more preferably from 10 000 to 100 000 approximately.

As grafted silicone polymers that may be used according to the present invention, mention may be made of the product sold by the company 3M under the reference VS80.

H) anionic polyurethanes.

The polyurethanes preferably used according to the invention preferably contain a base repeating unit corresponding to the formula (VIII) below:

—X′—B—X′—CO—NH—R—NH—CO— (VIII)

in which:
—X′ represents O and/or NH,
—B is a divalent hydrocarbon-based radical, this radical being substituted or unsubstituted, and
—R is a divalent radical chosen from branched or unbranched alkylene radicals of C₆-C₂₀ aromatic, C₁-C₂₀ and preferably C₁-C₆ aliphatic and C₁-C₂ and preferably C₁-C₆ cycloaliphatic type, these radicals being unsubstituted or substituted with one or more halogen, C₁-C₄ alkoxy or C₆-C₃₀ aryl, in particular phenyl, groups.

Preferably, the radical B is a C₁-C₃₀ and preferably C₂-C₁₀ divalent radical and bears a group containing one or more carboxylic functions and/or one or more sulfonic functions, the said carboxylic and/or sulfonic functions being in free form or partially or totally neutralized with a mineral or organic base such as alkali metal or alkaline-earth metal hydroxides, aqueous ammonia, alkylamines, alkanolamines or organic amino acids. B is preferably the divalent radical derived from dimethylolpropionic acid.

The radical R is advantageously chosen from the radicals corresponding to the following formulae:

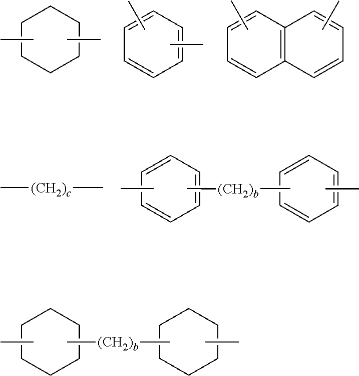

in which b is an integer between 0 and 3 and c is an integer between 1 and 20 and preferably between 2 and 12.

In particular, the radical R is chosen from hexamethylene, 4,4′-biphenylenemethane, 2,4- and/or 2,6-tolylene, 1,5-naphthylene, p-phenylene and methylene-4,4-bis-cyclohexyl radicals and the divalent radical derived from isophorone.

The fixing polyurethanes that may be used may comprise silicone grafts and silicones containing hydrocarbon-based grafts.

A polyurethane that may be used may also advantageously comprise at least one polysiloxane block, and its base repeating unit corresponds, for example, to the general formula (IX):

—X′—P—X′—CO—NH—R—NH—CO (IX)
in which:
P is a polysiloxane segment,
X′ represents O and/or NH, and
R is a divalent radical chosen from branched or unbranched alkylene radicals of C₆-C₂₀ aromatic, C₁-C₂₀ and preferably C₁-C₆ aliphatic and C₁-C₂₀ and preferably C₁-C₆ cycloaliphatic type, these radicals being unsubstituted or substituted with one or more halogen, C₁-C₄ alkoxy and C₁-C₃₀ aryl, in particular phenyl, groups.

The radical R is advantageously chosen from the radicals corresponding to the following formulae:

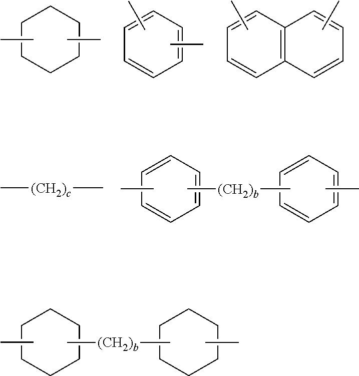

in which b is an integer between 0 and 3 and c is an integer between 1 and 20 and preferably between 2 and 12.

In particular, the radical R is chosen from hexamethylene, 4,4′-biphenylenemethane, 2,4- and/or 2,6-tolylene, 1,5-naphthylene, p-phenylene and methylene-4,4-bis-cyclohexyl radicals and the divalent radical derived from isophorone.

Advantageously, the polysiloxane segment P corresponds to the general formula (X) below:

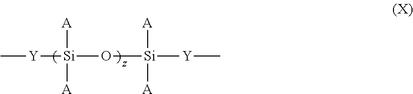

in which:
the groups A, which may be identical or different, are chosen firstly from C₁-C₂₀ monovalent hydrocarbon-based groups substantially free of ethylenic unsaturation and, secondly, from aromatic groups,
Y represents a divalent hydrocarbon-based group, and
Z represents an integer chosen such that the average molecular mass of the polysiloxane segment is between 300 and 10 000.

In general, the divalent group Y is chosen from the alkylene groups of formula —(CH₂)ₐ—, in which a represents an integer that may be between 1 and 10.

The groups A may be chosen from C₁-C₁₈ alkyl groups, in particular methyl, ethyl, propyl, isopropyl, butyl, pentyl, hexyl, octyl, decyl, dodecyl and octadecyl groups; cycloalkyl groups, in particular the cyclohexyl group; aryl groups, especially phenyl and naphthyl; arylalkyl groups, especially benzyl and phenylethyl, and also tolyl and xylyl groups.

Examples of fixing polyurethanes that may especially be mentioned include the dimethylolpropionic acid/isophorone diisocyanate/neopentyl glycol/polyester diols copolymer (also known under the name polyurethane-1, INCI name) sold under the brand name Luviset® Pur by the company BASF, and the dimethylolpropionic acid/isophorone diisocyanate/neopentyl glycol/polyester diols/silicone diamine copolymer (also known under the name polyurethane-6, INCI name) sold under the brand name Luviset® Si PUR A by the company BASF.

Another anionic polyurethane that may also be used is Avalure UR 450.

Polymers containing sulfoisophthalate groups, such as the polymers AQ55 and AQ48 sold by the company Eastman, may also be used.

According to the invention, the anionic polymers are preferably chosen from acrylic acid copolymers such as the acrylic acid/ethyl acrylate/N-tert-butylacrylamide terpolymer sold under the name Ultrahold Strong® by the company BASF, and methacrylic acid/ethyl acrylate copolymers, especially in aqueous dispersion, such as Luviflex Soft and Luvimer MAE sold by the company BASF, and butyl acrylate/acrylic acid/methacrylic acid branched block copolymers such as Fixate G100L sold by the company Lubrizol.

Crotonic acid-based copolymers may also be used, such as vinyl acetate/vinyl tert-butylbenzoate/crotonic acid terpolymers and crotonic acid/vinyl acetate/vinyl neododecanoate terpolymers sold under the name Resin 28-29-30 by the company National Starch, polymers derived from maleic, fumaric or itaconic acid or anhydride with vinyl esters, vinyl ethers, vinyl halides or phenylvinyl derivatives, acrylic acid and esters thereof, such as the methyl vinyl ether/monoesterified maleic anhydride copolymer sold under the name Gantrez® ES 425 by the company ISP, Luviset Si Pur, Mexomer PW, elastomeric or non-elastomeric anionic polyurethanes, polymers containing sulfoisophthalate groups, anionic grafted silicone polymers, and also Amerhold DR 25 and VS 80.

The cationic fixing film-forming polymers that can be used according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1 000 and 3 000 000.

Among these polymers, mention may be made more particularly of the following cationic polymers:

(1) Homopolymers or copolymers derived from acrylic or methacrylic esters or amides and comprising at least one of the units of the following formulae:

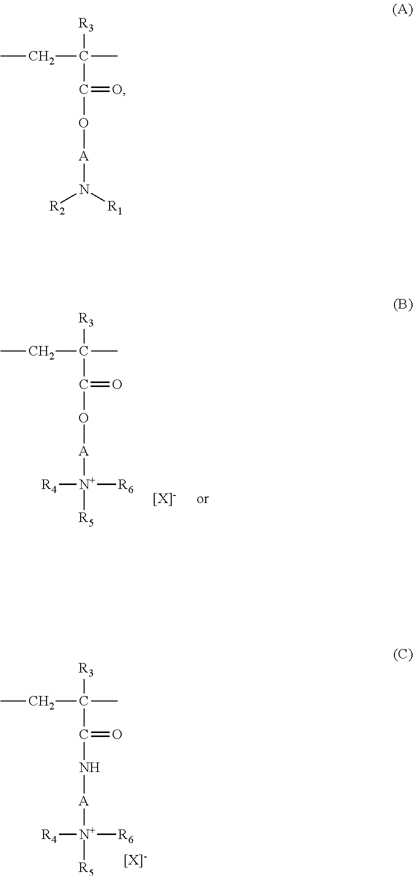

in which:
R₃ denotes a hydrogen atom or a CH₃ radical;
A is a linear or branched alkyl group comprising 1 to 6 carbon atoms or a hydroxyalkyl group comprising 1 to 4 carbon atoms;
R₄, R₅ and R₆, which are identical or different, represent an alkyl group containing from 1 to 6 carbon atoms or a benzyl group;
R₁ and R₂, which are identical or different, each represent a hydrogen atom or an alkyl group containing from 1 to 6 carbon atoms;
X denotes a methosulfate anion or a halide such as chloride or bromide.

The copolymers of the family (1) also contain one or more units derived from comonomers that may be chosen from the family of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc® by the company Hercules,
copolymers of acrylamide and of methacryloyloxyethyltrimethylammonium chloride, described, for example, in patent application EP-A-080 976 and sold under the name Bina Quat P 100 by the company Ciba Geigy,
copolymers of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat® by the company ISP, such as, for example, Gafquat® 734 or Gafquat® 755, or alternatively the products known as Copolymer® 845, 958 and 937. These polymers are described in detail in French patents 2 077 143 and 2 393 573,
fatty-chain polymers containing a vinylpyrrolidone unit, such as the products sold under the names Styleze W20 and Styleze W10 by the company ISP,
dimethylaminoethyl methacrylate/vinylcaprolactam/vinylpyrrolidone terpolymers, such as the product sold under the name Gaffix VC 713 by the company ISP, and
quaternized vinylpyrrolidone/dimethylaminopropylmethacrylamide copolymers, such as the products sold under the name Gafquat® HS 100 by the company ISP.

(2) Cationic guar gums, preferably containing quaternary ammonium, such as those described in U.S. Pat. Nos. 3,589,578 and 4,031,307, such as guar gums containing trialkylammonium cationic groups. Such products are sold in particular under the trade names Jaguar C13 S, Jaguar C 15 and Jaguar C 17 by the company Meyhall.

(3) Quaternary copolymers of vinylpyrrolidone and of vinylimidazole.

(4) Chitosans or salts thereof; the salts that can be used are, in particular, chitosan acetate, lactate, glutamate, gluconate or pyrrolidonecarboxylate.

Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol.

(5) Cationic cellulose derivatives such as copolymers of cellulose or of cellulose derivatives grafted with a water-soluble monomer comprising a quaternary ammonium, and disclosed in particular in U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethylammonium or dimethyldiallylammonium salt.

The products sold corresponding to this definition are, more particularly, the products sold under the name Celquat L 200 and Celquat H 100 by the company National Starch.

The amphoteric fixing polymers that can be used in accordance with the invention can be chosen from polymers comprising units B and C distributed randomly in the polymer chain, in which B denotes a unit derived from a monomer comprising at least one basic nitrogen atom and C denotes a unit derived from an acid monomer comprising one or more carboxylic or sulfonic groups, or alternatively B and C can denote groups derived from carboxybetaine or sulfobetaine zwitterionic monomers; B and C can also denote a cationic polymer chain comprising primary, secondary, tertiary or quaternary amine groups, in which at least one of the amine groups bears a carboxylic or sulfonic group connected via a hydrocarbon group or alternatively B and C form part of a chain of a polymer containing an ethylenedicarboxylic unit in which one of the carboxylic groups has been made to react with a polyamine comprising one or more primary or secondary amine groups.

The amphoteric polymers corresponding to the definition given above that are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and -acrylamides. Such compounds are described in U.S. Pat. No. 3,836,537.

The vinyl compound may also be a dialkyldiallylammonium salt such as diethyldiallylammonium chloride.

(2) polymers comprising units derived from:

a) at least one monomer chosen from acrylamides and methacrylamides substituted on the nitrogen with an alkyl group,

b) at least one acidic comonomer containing one or more reactive carboxylic groups, and

c) at least one basic comonomer such as acrylic and methacrylic acid esters containing primary, secondary, tertiary and quaternary amine substituents, and the product of quaternization of dimethylaminoethyl methacrylate with dimethyl or diethyl sulfate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, or maleic or fumaric acids or anhydrides. The preferred basic comonomers are aminoethyl, butylaminoethyl, N,N′-dimethylaminoethyl and N-tert-butylaminoethyl methacrylates. The copolymers whose CTFA (4th edition, 1991) name is octylacrylamide/acrylates/butylaminoethyl methacrylate copolymer, such as the products sold under the name Amphomer® or Lovocryl® 47 by the company National Starch, are particularly used.

(3) crosslinked and alkylated polyamino amides partially or totally derived from polyamino amides of general formula:

CO—R₄CO-z (XI)

in which R₄ represents a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of the said acids to a bis(primary) or bis(secondary) amine, and Z denotes a group derived from a bis(primary), mono- or bis(secondary) polyalkylene polyamine and preferably represents:

a) in proportions of from 60 to 100 mol %, the group:

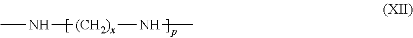

where x=2 and p=2 or 3, or alternatively x=3 and p=2

this group being derived from diethylenetriamine, from triethylenetetramine or from dipropylenetriamine;

b) in proportions of from 0 to 40 mol %, the group (XII) above in which x=2 and p=1 and which is derived from ethylenediamine, or the group derived from piperazine:

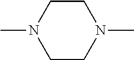

c) in proportions of from 0 to 20 mol %, the —NH—(CH₂)₆—NH— group being derived from hexamethylenediamine, these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid. The alkane sultones used in the acylation are preferably propane sultone or butane sultone, the salts of the acylating agents are preferably the sodium or potassium salts.

(4) polymers comprising zwitterionic units of formula:

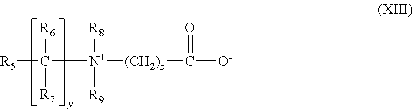

in which R₅ denotes a polymerizable unsaturated group such as an acrylate, methacrylate, acrylamide or methacrylamide group, y and z represent an integer from 1 to 3, R₆ and R₇ represent a hydrogen atom, a methyl, ethyl or propyl group, R₈ and R₉ represent a hydrogen atom or an alkyl group such that the sum of the carbon atoms in R₁₀ and R₁₁ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

(5) polymers derived from chitosan comprising monomer units corresponding to the following formulae:

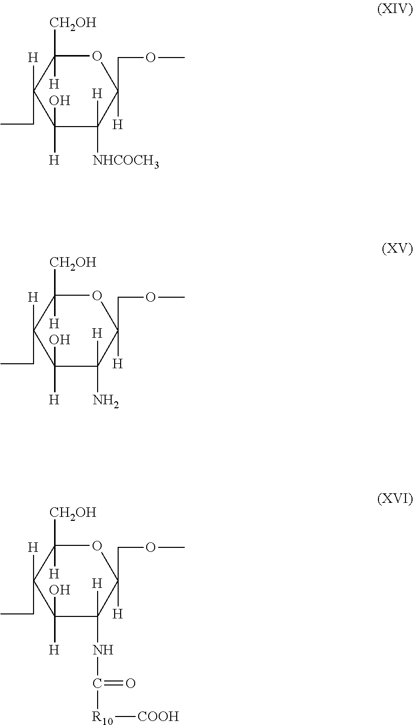

the unit (XIV) being present in proportions of between 0 and 30%, the unit (XV) in proportions of between 5% and 50% and the unit (XVI) in proportions of between 30% and 90%, it being understood that, in this unit F, R₁₀ represents a group of formula:

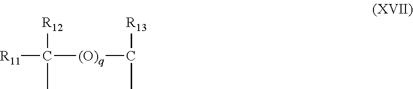

in which, if q=0, R₁₁, R₁₂ and R₁₃, which are identical or different, each represent a hydrogen atom, a methyl, hydroxyl, acetoxy or amino residue, a monoalkylamine residue or a dialkylamine residue that are optionally interrupted with one or more nitrogen atoms and/or optionally substituted by one or more amine, hydroxyl, carboxyl, alkylthio or sulfonic groups, an alkylthio residue in which the alkyl group bears an amino residue, at least one of the groups R₁₇, R₁₈ and R₁₉ being, in this case, a hydrogen atom;

or, if q=1, R₁₁, R₁₂ and R₁₃ each represent a hydrogen atom, as well as the salts formed by these compounds with bases or acids.

(6) polymers derived from the N-carboxyalkylation of chitosan.

(7) polymers of units corresponding to the general formula (XVIII) described, for example, in French patent 1 400 366:

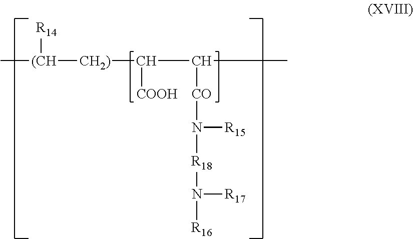

in which R₁₄ represents a hydrogen atom, a CH₃O, CH₃CH₂O or phenyl group, R₁₅ denotes a hydrogen atom or a lower alkyl group such as methyl or ethyl, R₁₆ denotes a hydrogen atom or a lower alkyl group such as methyl or ethyl, R₁₇ denotes a lower alkyl group such as methyl or ethyl or a group corresponding to the formula: —R₁₈—N(R₁₆)₂, R₁₈ representing a —CH₂—CH₂—, —CH₂—CH₂—CH₂— or —CH₂—CH(CH₃)— group, and R₁₆ having the abovementioned meanings,

and also the higher homologues of these groups, containing up to 6 carbon atoms.

(8) amphoteric polymers of the type -D-X-D-X— chosen from:

a) polymers obtained by the action of chloroacetic acid or sodium chloroacetate on compounds comprising at least one unit of formula:

-D-X-D-X-D- (XIX)

where D denotes a group

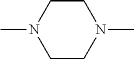

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) Polymers of formula:

-D-X-D-X— (XX)

where D denotes a group

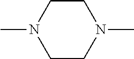

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

According to one preferred embodiment of the invention, the amphoteric fixing polymers that may be used in the process according to the invention may be chosen from branched block copolymers comprising:

(a) nonionic units derived from at least one monomer chosen from C₁-C₆ alkyl(meth)acrylates, N-mono(C₂-C₆ alkyl)(meth)acrylamides and N,N-di(C₂-C₆ alkyl)(meth)acrylamides,

(b) anionic units derived from at least one monomer chosen from acrylic acid and methacrylic acid, and

(c) polyfunctional units derived from at least one monomer comprising at least two polymerizable unsaturated functional groups,

and preferably having a structure consisting of hydrophobic blocks onto which are fixed, via polyfunctional units (c), several more hydrophilic blocks.

Preferably, the amphoteric polymers have at least two glass transition temperatures (Tg), of which at least one is greater than 20° C. and the other is less than 20° C.

The preferred amphoteric polymers are polymers comprising units derived from:

a) at least one monomer chosen from acrylamides and methacrylamides substituted on the nitrogen with an alkyl group,

b) at least one acidic comonomer containing one or more reactive carboxylic groups, and

c) at least one basic comonomer such as acrylic and methacrylic acid esters containing primary, secondary, tertiary and quaternary amine substituents, and the product of quaternization of dimethylaminoethyl methacrylate with dimethyl or diethyl sulfate.

Mention may be made in particular of the polymers sold under the name Amphomer by the company National Starch.

The nonionic polymers that may be used according to the present invention are chosen, for example, from:
vinylpyrrolidone homopolymers;
polyalkyloxazolines;
vinyl acetate homopolymers;
vinyl acetate copolymers, for instance copolymers of vinyl acetate and of acrylic ester; copolymers of vinyl acetate and of ethylene, or copolymers of vinyl acetate and of maleic ester, for example of dibutyl maleate;
acrylic ester homopolymers and copolymers, for instance copolymers of alkyl acrylates and of alkyl methacrylates, such as the products sold by Rohm & Haas under the names Primal® AC-261 K and Eudragit® NE 30 D, by BASF under the name 8845, or by Hoechst under the name Appretan® N9212;
copolymers of acrylonitrile and of a nonionic monomer chosen, for example, from butadiene and alkyl(meth)acrylates; mention may be made of the products sold under the name CJ 0601 B by the company Rohm & Haas;
styrene homopolymers;
styrene copolymers, for instance copolymers of styrene and of an alkyl(meth)acrylate, such as the products Mowilith® LDM 6911, Mowilith® DM 611 and Mowilith® LDM 6070 sold by Hoechst, and the products Rhodopas® SD 215 and Rhodopas® DS 910 sold by Rhodia Chimie; copolymers of styrene, of alkyl methacrylate and of alkyl acrylate; copolymers of styrene and of butadiene; or copolymers of styrene, of butadiene and of vinylpyridine;
polyamides;
vinyllactam homopolymers, such as the polyvinylcaprolactam sold under the name Luviskol® Plus by the company BASF; and vinylpyrrolidone homopolymers;
vinyllactam copolymers such as a poly(vinylpyrrolidone/vinyllactam) copolymer sold under the trade name Luvitec® VPC 55K65W by BASF, poly(vinylpyrrolidone/vinyl acetate) copolymers, such as those sold under the name PVPVA® S630L by ISP, Luviskol® VA 73, VA 64, VA 55, VA 37 and VA 28 by BASF; and poly(vinylpyrrolidone/vinyl acetate/vinyl propionate) terpolymers, for instance the product sold under the name Luviskol® VAP 343 by BASF.

The alkyl groups of the nonionic polymers mentioned above preferably contain from 1 to 6 carbon atoms.

Preferably, the fixing polymer(s) of the invention are chosen from anionic and nonionic polymers and even more preferentially from anionic polymers.

The concentration of fixing polymer(s) in the compositions of the invention preferably ranges from 0.1% to 10%, preferably from 0.3% to 8% and better still from 0.5% to 5% by weight relative to the total weight of the composition.

Associative Polymer

Associative polymers are water-soluble polymers that are capable, in an aqueous medium, of reversibly combining with each other or with other molecules.

Their chemical structure comprises one or more hydrophilic regions and one or more hydrophobic regions characterized by the presence of at least one C8-C30 fatty chain.

Associative polymers may be of anionic, cationic, amphoteric or nonionic type.

Among the associative polymers of anionic type that may be mentioned are:
(I) those comprising at least one hydrophilic unit and at least one fatty-chain allyl ether unit, more particularly those whose hydrophilic unit consists of an ethylenic unsaturated anionic monomer, more particularly of a vinylcarboxylic acid and most particularly of an acrylic acid or a methacrylic acid or mixtures thereof, the fatty-chain allyl ether unit of which corresponds to the monomer of formula (XXI) below:

CH₂═CR′CH₂OBₙR (XXI)

in which R′ denotes H or CH₃, B denotes an ethylenoxy radical, n is zero or denotes an integer ranging from 1 to 100, R denotes a hydrocarbon-based radical chosen from alkyl, arylalkyl, aryl, alkylaryl and cycloalkyl radicals, comprising from 8 to 30 carbon atoms, preferably 10 to 24 and even more particularly from 12 to 18 carbon atoms. The unit of formula (XXI) that is more particularly preferred is a unit in which R′ denotes H, n is equal to 10, and R denotes a stearyl radical (C₁₈).

Anionic associative polymers of this type are described and prepared, according to an emulsion polymerization process, in patent EP-0 216 479.

Among these anionic associative polymers that are particularly preferred according to the invention are polymers formed from 20% to 60% by weight of acrylic acid and/or of methacrylic acid, from 5% to 60% by weight of lower alkyl(meth)acrylates, from 2% to 50% by weight of fatty-chain allyl ether of formula (XXI), and from 0 to 1% by weight of a crosslinking agent which is a well-known copolymerizable unsaturated polyethylenic monomer, for instance diallyl phthalate, allyl(meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), in particular those sold by the company Allied Colloids under the names Salcare SC 80® and Salcare SC 90®, which are aqueous 30% emulsions of a crosslinked terpolymer of methacrylic acid, of ethyl acrylate and of steareth-10 allyl ether (40/50/10).
(II) polymers comprising at least one hydrophilic unit of unsaturated olefinic carboxylic acid type, and at least one hydrophobic unit of the type such as a (C₁₀-C₃₀) alkyl ester of an unsaturated carboxylic acid.

These polymers are preferably chosen from those in which the hydrophilic unit of unsaturated olefinic carboxylic acid type corresponds to the monomer of formula (XXII) below:

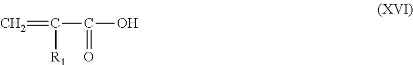

Formula number to be modified to (XXII)

in which R₁ denotes H or CH₃ or C₂H₅, i.e. acrylic acid, methacrylic acid or ethacrylic acid units, and whose hydrophobic unit of the type such as a (C₁₀-C₃₀) alkyl ester of an unsaturated carboxylic acid corresponds to the monomer of formula (XXIII) below:

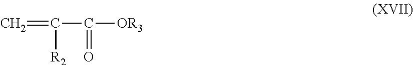

Formula number to be modified to (XXIII)

in which R₂ denotes H or CH₃ or C₂H₅ (i.e. acrylate, methacrylate or ethacrylate units) and preferably H (acrylate units) or CH₃ (methacrylate units), R₃ denoting a C₁₀-C₃₀ and preferably C₁₂-C₂₂ alkyl radical.

(C₁₀-C₃₀)alkyl esters of unsaturated carboxylic acids in accordance with the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. No. 3,915,921 and U.S. Pat. No. 4,509,949.

Among the anionic associative polymers of this type that will be used more particularly are polymers formed from a monomer mixture comprising:

(i) essentially acrylic acid,

(ii) an ester of formula (XXIII) described above and in which R₂ denotes H or CH₃, R₃ denoting an alkyl radical containing from 12 to 22 carbon atoms, and

(iii) a crosslinking agent, which is a well-known copolymerizable unsaturated polyethylenic monomer, for instance diallyl phthalate, allyl(meth)acrylate, divinylbenzene, (poly)ethylene glycol dimethacrylate and methylenebisacrylamide.

Among the anionic associative polymers of this type, use will be made more particularly of those formed from 95% to 60% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those formed from 98% to 96% by weight of acrylic acid (hydrophilic unit), 1% to 4% by weight of C₁₀-C₃₀ alkyl acrylate

(hydrophobic unit) and 0.1% to 0.6% by weight of crosslinking polymerizable monomer such as those described previously.

Among the said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.
(III) maleic anhydride/C₃₀-C₃₈ α-olefin/alkyl maleate terpolymers, such as the product (maleic anhydride/C₃₀-C₃₈ α-olefin/isopropyl maleate) sold under the name Performa V 1608® by the company Newphase Technologies.
(IV) acrylic terpolymers comprising:

(a) about 20% to 70% by weight of a carboxylic acid containing α,β-monoethylenic unsaturation,

(b) about 20% to 80% by weight of a non-surfactant monomer containing α,β-monoethylenic unsaturation other than (a),

(c) about 0.5% to 60% by weight of a nonionic monourethane which is the product of reaction of a monohydric surfactant with a monoisocyanate containing monoethylenic unsaturation,

such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(V) copolymers comprising among their monomers a carboxylic acid containing α,β-monoethylenic unsaturation and an ester of a carboxylic acid containing α,β-monoethylenic unsaturation and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer.

Among the associative polymers of cationic type that may be mentioned are:
(I) cationic associative polyurethanes, the family of which has been described by the Applicant in French patent application No. 00/09609; it may be represented by the general formula (XXIV) below:

R—X—(P)n-[L-(Y)m]r-L′-(P′)p-X′—R′ (XXIV)

in which:

R and R′, which may be identical or different, represent a hydrophobic group or a hydrogen atom;

X and X′, which may be identical or different, represent a group comprising an amine function optionally bearing a hydrophobic group, or alternatively a group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100, preferably between 1 and 50 and in particular between 1 and 25;

n, m and p each range, independently of each other, from 0 to 1000;

the molecule containing at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (XXIV) described above and in which:

R and R′ both independently represent a hydrophobic group,
X and X′ each represent a group L″,
n and p are between 1 and 1000, and
L, L′, L″, P, P′, Y and m have the meaning given above.

Another preferred family of cationic associative polyurethanes is the one corresponding to formula (XXIV) above in which:

R and R′ both independently represent a hydrophobic group, X and X′ each represent a group L″, n and p are 0, and L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Yet another preferred family of cationic associative polyurethanes is the one corresponding to formula (Ia) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500 000, in particular between 1000 and 400 000 and ideally between 1000 and 300 000.

The expression “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it comprises at least 10 carbon atoms, preferably from 10 to 30 carbon atoms, in particular from 12 to 30 carbon atoms and more preferably from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group comprising a tertiary or quaternary amine, X and/or X′ may represent one of the following formulae:

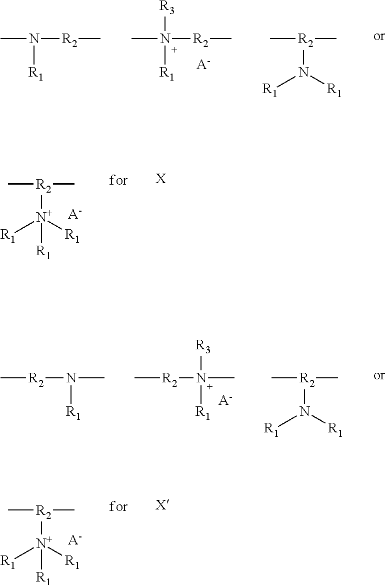

in which:

R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;

R₁ et R₃, which may be identical or different, denote a C₁-C₃₀ alkyl or alkenyl radical or an aryl radical, at least one of the carbon atoms possibly being replaced with a heteroatom chosen from N, S, O and P;

A⁻ is a physiologically acceptable counterion.

The groups L, L′ and L″ represent a group of formula:

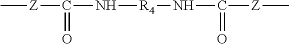

in which:

Z represents —O—, —S— or —NH—; and
R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

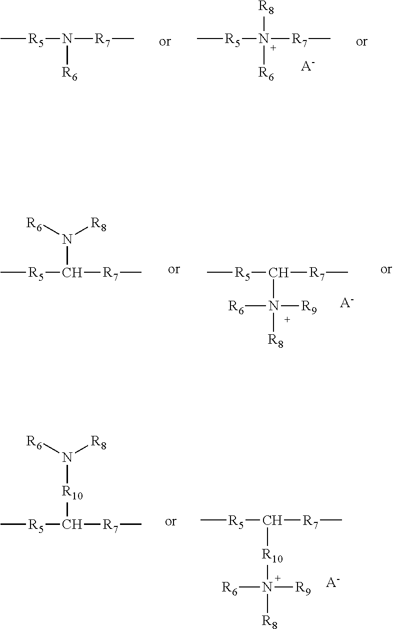

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more heteroatoms chosen from N, O, S and P; and

A⁻ is a physiologically acceptable counterion.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and especially a poly(ethylene oxide) or poly(propylene oxide).

The cationic associative polyurethanes of formula (XXIV) that may be used according to the invention are formed from diisocyanates and from various compounds with functions containing a labile hydrogen. The functions containing a labile hydrogen may be alcohol, primary or secondary amine or thiol functions, giving, after reaction with the diisocyanate functions, polyurethanes, polyureas and polythioureas, respectively. The expression “polyurethanes” that may be used according to the present invention encompasses these three types of polymer, namely polyurethanes per se, polyureas and polythioureas, and also copolymers thereof.

A first type of compound involved in the preparation of the polyurethane of formula (XXIV) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)n-ZH

or

HZ—(P′)p-ZH

in which Z, P, P′, n and p are as defined above.

Examples of compounds containing an amine function that may be mentioned include N-methyldiethanolamine, N-tert-butyldiethanolamine and N-sulfoethyl diethanolamine.

The second compound included in the preparation of the polyurethane of formula (XVIII) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (XXIV) is a hydrophobic compound intended to form the terminal hydrophobic groups of the polymer of formula (XXIV).

This compound consists of a hydrophobic group and of a function containing a labile hydrogen, for example a hydroxyl, primary or secondary amine, or thiol function.

By way of example, this compound may be a fatty alcohol such as, in particular, stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (XXIV) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and especially a poly(ethylene oxide) or poly(propylene oxide).

The hydrophilic group termed Y in formula (XXIV) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(II) quaternized cellulose derivatives and polyacrylates containing non-cyclic amine side groups.

The quaternized cellulose derivatives are, in particular:
quaternized celluloses modified with groups comprising at least one fatty chain, such as alkyl, arylalkyl or alkylaryl groups comprising at least 8 carbon atoms, or mixtures thereof,
quaternized hydroxyethylcelluloses modified with groups comprising at least one fatty chain, such as alkyl, arylalkyl or alkylaryl groups comprising at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl or anthryl groups.

Examples of quaternized alkylhydroxyethylcelluloses containing C₈-C₃₀ fatty chains that may be mentioned include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol, and the products Crodacel QM®, Crodacel QUO (C₁₂ alkyl) and Crodacel QS® (C₁₈ alkyl) sold by the company Croda.

The amphoteric associative polymers are preferably chosen from those comprising at least one non-cyclic cationic unit. Even more particularly, the ones that are preferred are those prepared from or comprising 1 to 20 mol %, preferably 1.5 to 15 mol % and even more particularly 1.5 to 6 mol % of fatty-chain monomer relative to the total number of moles of monomers.

The amphoteric associative polymers that are preferred according to the invention comprise or are prepared by copolymerizing:

1) at least one monomer of formula (XXV) or (XXVI):

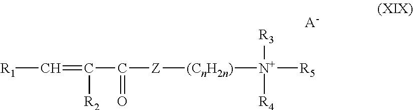

Formula number to be modified to (XXV)

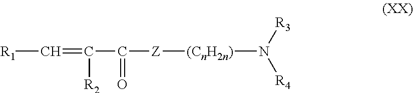

Formula number to be modified to (XXVI)

in which R₁ and R₂, which may be identical or different, represent a hydrogen atom or a methyl radical, R₃, R₄ and R₅, which may be identical or different, represent a linear or branched alkyl radical containing from 1 to 30 carbon atoms,

Z represents an NH group or an oxygen atom,

n is an integer from 2 to 5,

A⁻ is an anion derived from an organic or mineral acid, such as a methosulfate anion or a halide such as chloride or bromide;

2) at least one monomer of formula (XXVII)

R₆—CH═CR₇—COOH (XXI)
Formula number to be modified to (XXVII)

in which R₆ and R₇, which may be identical or different, represent a hydrogen atom or a methyl radical;

and

3) at least one monomer of formula (XXVIII)

R₆—CH═CR₇—COXR₈ (XXII)
Formula number to be modified to (XXVIII)

in which R₆ and R₇, which may be identical or different, represent a hydrogen atom or a methyl radical, X denotes an oxygen or nitrogen atom and R₈ denotes a linear or branched alkyl radical containing from 1 to 30 carbon atoms;

at least one of the monomers of formula (XXV), (XXVI) or (XXVIII) comprising at least one fatty chain.

The monomers of formulae (XXV) and (XXVI) of the present invention are preferably chosen from the group consisting of:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate,

dimethylaminopropylmethacrylamide, dimethylaminopropylacrylamide,

these monomers optionally being quaternized, for example with a C₁-C₄ alkyl halide or a C₁-C₄ dialkyl sulfate.

More particularly, the monomer of formula (XXV) is chosen from acrylamido-propyltrimethylammonium chloride and methacrylamidopropyltrimethylammonium chloride.

The monomers of formula (XXVII) of the present invention are preferably chosen from the group consisting of acrylic acid, methacrylic acid, crotonic acid and 2-methylcrotonic acid. More particularly, the monomer of formula (XXI) is acrylic acid.

The monomers of formula (XXVIII) of the present invention are preferably chosen from the group formed from C₁₂-C₂₂ and more particularly C₁₆-C₁₈ alkyl acrylates or methacrylates.

The monomers constituting the fatty-chain amphoteric polymers of the invention are preferably already neutralized and/or quaternized.

The ratio of the number of cationic charges/anionic charges is preferably equal to about 1.

The amphoteric associative polymers according to the invention preferably comprise from 1 mol % to 10 mol % of the monomer comprising a fatty chain (monomer of formula (XXV), (XXVI) or (XXVIII)), and preferably from 1.5 mol % to 6 mol %.

The weight-average molecular weights of the amphoteric associative polymers according to the invention may range from 500 to 50 000 000 and are preferably between 10 000 and 5 000 000.

The amphoteric associative polymers according to the invention may also contain other monomers such as nonionic monomers and in particular such as C₁-C₄ alkyl acrylates or methacrylates.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:
(1) celluloses modified with groups comprising at least one fatty chain;

examples that may be mentioned include:
hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS® (C₁₆ alkyl) sold by the company Aqualon, or the product Bermocoll EHM 100® sold by the company Berol Nobel,
hydroxyethylcelluloses modified with alkylphenyl polyalkylene glycol ether groups, such as the product Amercell Polymer HM-1500® (nonylphenyl polyethylene glycol (15) ether) sold by the company Amerchol.
(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhone-Poulenc.
(3) copolymers of vinylpyrrolidone and of fatty-chain hydrophobic monomers; examples that may be mentioned include:
the products Antaron V216® or Ganex V216® (vinylpyrrolidone/hexadecene copolymer) sold by the company ISP.
the products Antaron V220® or Ganex V220® (vinylpyrrolidone/eicosene copolymer) sold by the company ISP.
(4) copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name Antil 208®.
(5) copolymers of hydrophilic methacrylates or acrylates and of hydrophobic monomers comprising at least one fatty chain, for instance the polyethylene glycol methacrylate/lauryl methacrylate copolymer.
(6) polyurethane polyethers comprising in their chain both hydrophilic blocks usually of polyoxyethylenated nature and hydrophobic blocks, which may be aliphatic sequences alone and/or cycloaliphatic and/or aromatic sequences.
(7) polymers with an aminoplast ether backbone containing at least one fatty chain, such as the Pure Thix® compounds sold by the company Sud-Chemie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane linkage between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂₋₁₄ alkyl chain, and the product Elfacos T212® containing a C₁₈ alkyl chain, from Akzo.

The product DW 1206B® from Rohm & Haas containing a C₂₀ alkyl chain and a urethane bond, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Röhm & Haas may also be used.

The polyurethane polyethers that may be used according to the invention are in particular those described in the article by G. Fonnum, J. Bakke and Fk. Hansen—Colloid Polym. Sci 271, 380.389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

According to one particularly preferred variant, the composition according to the present patent application comprises one or more associative polymers of anionic type.

The concentration of associative polymer(s) in the compositions of the invention preferably ranges from 0.1% to 10%, preferably from 0.3% to 8% and better still from 0.5% to 5% by weight relative to the total weight of the composition.

Antidandruff Agent

The antidandruff agents that may be used according to the invention are preferably chosen from:

1) pyridinethione salts, especially the calcium, magnesium, barium, strontium, zinc, cadmium, tin and zirconium salts. The zinc salt of pyridinethione is particularly preferred.

The zinc salt of pyridinethione is sold especially under the name Omadine zinc by the company Arch Personal Care.

2) 1-hydroxy-2-pyrrolidone derivatives represented especially by formula (XXIX):

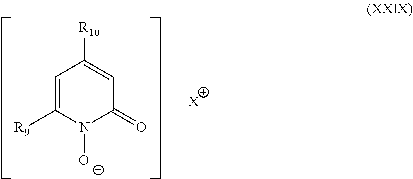

in which R9 represents an alkyl group containing from 1 to 17 carbon atoms, an alkenyl group containing from 2 to 17 carbon atoms, a cycloalkyl group containing from 5 to 8 carbon atoms, a bicycloalkyl group containing from 7 to 9 carbon atoms; a cycloalkyl (-alkyl) group, an aryl group, an aralkyl group with an alkyl containing from 1 to 4 carbon atoms, an arylalkenyl group with an alkenyl containing from 2 to 4 carbon atoms, aryloxyalkyl or arylmercaptoalkyl with an alkyl containing from 1 to 4 carbon atoms, a furylalkenyl group with an alkenyl or a furyl containing from 2 to 4 carbon atoms, an alkoxy group containing from 1 to 4 carbon atoms, a nitro group, a cyano group or a halogen atom;

R10 represents a hydrogen atom, a C1-C4 alkyl group, a C2-C4 alkenyl group, a halogen atom, a phenyl group, a benzyl group; Y represents an organic base, an alkali metal or alkaline-earth metal ion or an ammonium ion.

Examples of compounds of formula (XXIX) include 1-hydroxy-4-methyl-2-pyridone, 1-hydroxy-6-methyl-2-pyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methylcyclohexyl)-2-pyridone, 1-hydroxy-4-methyl-6-(2-bicyclo[2,2,1]heptyl)-2-pyridone, 1-hydroxy-4-methyl-6-(4-methylphenyl)-2-pyridone, 1-hydroxy-4-methyl-6-[1-(4-nitrophenoxy)butyl]-2-pyridone, 1-hydroxy-4-methyl-6-(4-cyanophenoxymethyl)-2-pyridone, 1-hydroxy-4-methyl-6-(phenyl sulfonylmethyl)-2-pyridone and 1-hydroxy-4-methyl-6-(4-bromobenzyl)-2-pyridone.

The compounds of formula (XXIX) may be used in the form of salts with organic or mineral bases.

Examples of organic bases are especially alkanolamines of low molecular weight such as ethanolamine, diethanolamine, N-ethylethanolamine, triethanolamine, diethylaminoethanol and 2-amino-2-methylpropanediol; non-volatile bases such as ethylenediamine, hexamethylenediamine, cyclohexylamine, benzylamine and N-methylpiperazine; quaternary ammonium hydroxides, e.g. trimethylbenzyl hydroxide; guanidine and derivatives thereof, and particularly alkyl derivatives thereof. Examples of mineral bases are especially salts of alkali metals, e.g. sodium or potassium; ammonium salts, salts of an alkaline-earth metal, e.g. magnesium or calcium; salts of di-, tri- or tetravalent cationic metals, e.g. zinc, aluminium or zirconium. Alkanolamines, ethylenediamine and mineral bases such as alkali metal salts are preferred.

A compound of formula (XXIX) that is particularly preferred is the one for which R9 denotes the radical

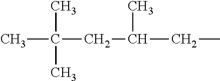

R10 denotes methyl

and X+ denotes N+H3CH2CH2OH

This compound is sold, for example, under the name Octopirox (1-hydroxy-4-methyl-6-(2,4,4-trimethylpentyl)-2-pyridone, monoethanolamine salt) by the company Hoechst.

3) 2,2′-dithiobis(pyridine N-oxide) of formula (XXX):

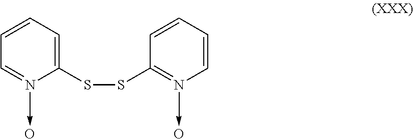 [SMILES: [O-][n+]1ccccc1SSc1cccc[n+]1[O-]]

The compounds of formula (XXX) may be introduced into the compositions in the form of mineral salts. An example of a mineral salt is magnesium sulfate.

4) the trihalocarbamides especially of formula (XXXI) below:

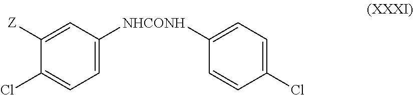

in which Z represents a halogen atom such as chlorine or a C1-C4 trihaloalkyl group such as CF3;

5) triclosan, represented by formula (XXXII):

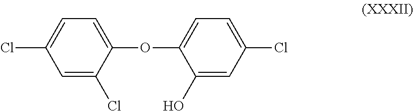 [SMILES: Oc1cc(Cl)ccc1Oc1ccc(Cl)cc1Cl]

6) azole compounds such as climbazole, ketoconazole, clotrimazole, econazole, isoconazole and miconazole.

7) selenium sulfides, in particular those of formula SxSe8-x, x ranging from 1 to 7.

8) extracts of one or more non-photosynthetic, non-fruiting filamentous bacteria.

The bacterial extracts that may be used according to the invention will be chosen from non-photosynthetic, non-fruiting filamentous bacteria as defined according to the classification in Bergey's Manual of Systemic Bacteriology, volume 3, section 23, 9th edition 1989.

Among the bacteria that may be used, mention will be made more particularly of bacteria belonging to the order Beggiatoales, and especially bacteria belonging to the genus Beggiotoa, for instance various strains of Beggiotoa alba. According to the definition, B. alba corresponds to the former names Beggiotoa arachnoidea, B. gigantea, B. leptomiformis, B. minima and B. mirabilis of Bergey's manual, 8th edition. Mention may moreover be made of bacteria belonging to the genus Vitreoscilla, which is known to be close to and often difficult to distinguish from the genus Beggiatoa. The bacteria that have just been defined, and several of which have been described, generally have an aquatic habitat, and may be found especially in spring water sources.

Among the bacteria that may be used, mention may be made, for example, of Vitreoscilla beggiatoides (ATCC 43181) and Beggiatoa alba (ATCC33555).

Preferentially, according to the invention, the use of the extract of Vitreoscilla filiformis, in particular the strain ATCC 15551, metabolites thereof and fractions thereof, are claimed.

Moreover, it is known that culturing non-photosynthetic, non-fruiting filamentous bacteria is relatively difficult, as is the production of pure cultures. Use will preferentially be made of the culture described in patent application WO 94/02158.

The term “non-photosynthetic, non-fruiting filamentous bacteria” means not only the culture supernatant but also the biomass obtained after culturing the said bacteria, the envelopes or envelope fractions, or the extracts of the biomass obtained by treating this biomass.

To prepare the extract according to the invention, the said bacteria can be cultured and then separated from the biomass obtained, for example by filtration, centrifugation, coagulation and/or lyophilization.

The extracts that may be used may especially be prepared according to the process described in patent application WO-A-93/00741. Thus, after culturing, the bacteria are concentrated by centrifugation. The biomass obtained is autoclaved. This biomass may be lyophilized to constitute what is known as the lyophilized extract. Any lyophilization method known to those skilled in the art may be used to prepare this extract.

The supernatant fraction of this biomass may also be filtered in a sterile container to remove the particles in suspension.

The terms “envelopes” and “envelope fractions” refer herein to the bacterial wall and possibly the subjacent membranes.

9) antifungal polymers such as amphotericin B or nystatin.

10) other antidandruff agents are sulfur in its various forms, cadmium sulfide, allantoin, coal or wood tars and derivatives thereof, in particular cade oil, salicylic acid, undecylenic acid, fumaric acid, ellagic acid, ellagic acid tannins, and allylamines such as terbinafine.

Preferably, the antidandruff agents of the invention contain one or more sulfur atoms in their structure.

Omadine zinc, 1-hydroxy-4-methyl-6-(2,4,4-trimethylpentyl)-2-pyridone and salts thereof and selenium sulfides are particularly preferred.

Preferably, the antidandruff agent is soluble in the composition of the invention at a temperature of 25° C. and at atmospheric pressure (760 mmHg).

Even more preferentially, it is soluble in water to a concentration of greater than or equal to 0.2% at a temperature of 25° C. and at atmospheric pressure (760 mmHg).

1-Hydroxy-4-methyl-6-(2,4,4-trimethylpentyl)-2-pyridone and salts thereof are particularly preferred.

The antidandruff agent(s) (iii) may be present in a content ranging from 0.001% to 10% by weight, preferably ranging from 0.1% to 5% by weight and more preferentially from 0.2% to 2% by weight relative to the total weight of the composition of the invention.

Cosmetic Medium

The term “cosmetically acceptable medium” means a medium that is compatible with keratin materials.

This cosmetically acceptable medium may comprise water, a mixture of one or more organic solvents or a mixture of water and of one or more organic solvents, the organic solvents preferably being chosen from C1-C4 lower alcohols, such as ethanol, isopropanol, tert-butanol or n-butanol; polyols such as glycerol, propylene glycol and polyethylene glycols; liquid linear or branched alkanes such as isohexadecane; and mixtures thereof.

The concentration of organic solvents in the compositions according to the present invention is between 0 and 99% and preferably between 0 and 95% by weight relative to the total weight of the composition.

In one variant of the invention, the compositions according to the invention contain less than 10% by weight of water and are optionally anhydrous, i.e. they contain no added water, the only water present being the water of crystallization of the starting materials.

In another variant of the invention, the compositions according to the invention contain one or more C1-C4 alcohols, preferentially ethanol.

The concentration of C1-C4 alcohol in the compositions according to the present invention is then between 0.1% and 99%, preferably between 1% and 95% and even more preferably between 20% and 80% by weight relative to the total weight of the composition.

Additional organic solvents that may be used in the compositions according to the present invention include polyols such as propylene glycol, polyethylene glycols and polyol ethers, and mixtures thereof.

The composition may also contain one or more additives other than the compounds (i), (ii) and (iii) of the invention, chosen from the active principles and cosmetic adjuvants commonly used in the field of haircare. These additives are chosen, for example, from vitamins, amino acids, oligopeptides, peptides, hydrolysed or non-hydrolysed, modified or unmodified proteins, enzymes, branched or unbranched fatty acids and fatty alcohols, animal, plant or mineral waxes, ceramides and pseudoceramides, hydroxylated organic acids, UV-screening agents, antioxidants and free-radical scavengers, chelating agents, seborrhoea regulators, calmatives, ionic or nonionic surfactants, silicones, mineral, plant or animal oils, polyisobutenes and poly(α-olefins), fatty esters, thickeners other than the polymers of the invention and in particular hydroxyethylcelluloses, hair dyes such as direct dyes, oxidation dye precursors and pigments, acids, bases, plasticizers, fragrances, preserving agents, mineral fillers, nacres and glitter flakes.

These additives are present in the composition according to the invention in an amount ranging from 0 to 20% by weight relative to the total weight of the composition.

Needless to say, a person skilled in the art will take care to select this or these additional active principles and cosmetic adjuvants such that the advantageous properties intrinsically associated with the compositions in accordance with the invention are not, or are not substantially, adversely affected by the envisaged addition(s).

The compositions according to the invention may be in the form of a rinse-out or leave-in care composition, and may be in the form of a thickened lotion, a cream, a gel or an emulsion.

As indicated previously, preferably, the compositions of the invention are in the form of gels.

Preferably, the compositions have a viscosity of greater than or equal to 4 Pa·s and better still ranging from 4 Pa·s to 500 Pa·s at a temperature of 25° C. and at a shear rate of 1 s⁻¹ (measurable, for example, with a Haake RS600 rheometer).

Another subject of the invention is the use of the cosmetic composition as described above for the cosmetic treatment of keratin materials, preferably the hair and the scalp. Even more preferentially, another subject of the invention is the use of the cosmetic composition as described above for the antidandruff treatment of keratin materials, preferably the hair and the scalp.

The invention also relates to a cosmetic process for treating keratin materials, preferably human keratin materials such as the hair and the scalp, which comprises the application of an effective amount of a cosmetic composition as described above to the said materials, and optional rinsing of the said composition after an optional leave-on time.

When the composition according to the invention is applied, it is optionally left on to act for about 0.5 to 5 minutes, and is then optionally rinsed out with water.

The application of the composition according to the invention may take place in the presence of heat at a temperature of between 60 and 220° C. by means of any heating device, for instance a hairdryer, a hood or an iron.

The examples that follow are given as illustrations of the present invention.

In the examples that follow, all the amounts are indicated as weight percentages of product in unmodified form relative to the total weight of the composition, unless otherwise indicated.

The example that follows serves to illustrate the invention without, however, being limiting in nature.

### EXAMPLE

In the example that follows, all the amounts are indicated as weight percentages of product in unmodified form relative to the total weight of the composition.

The following composition was prepared:

| | |
|---|---|
| Commercial reference | Amount |

| | | | | |
|---|---|---|---|---|
| Fragrance | | | 0.3 | |
| 1-Hydroxy-4-methyl-6- | OCTOPIROX | | 0.25 | |
| trimethylpentyl-2-pyridone, | (CLARIANT) | | | |
| monoethanolamine salt | | | | |
| 96° denatured ethyl alcohol | Alcohol | | 30 | |
| Guar gum, 2-hydroxypropyl | JAGUAR HP 105 | | 0.05 | |
| ether | (RHODIA) | | | |
| Propylene glycol | | | 3 | |
| Acrylates/C10-30 Alkyl acrylate | CARBOPOL ULTREZ 21 | | 1.6 | |
| crosspolymer | POLYMER (LUBRIZOL) | | | |
| Glycerol | | | 3 | |
| Sorbitol as an aqueous 70% | | | 3 | |
| solution | | | | |
| Butyl acrylate/acrylic | FIXATE G100L | | 12 | |
| acid/methacrylic acid branched | POLYMER (ISP) | | | |
| block polymer in water | | | | |
| 2-Amino-2-methyl-1-propanol | | | 1.4 | |
| Water | | Qs | 100 | g |

This composition is a clear composition that is easy to apply to the hair without leaving any residue.

The antidandruff efficacy is very satisfactory.

1.-15. (canceled)

16. A cosmetic composition comprising, in a cosmetically acceptable medium:
(i) at least one fixing polymer;
(ii) at least one associative polymer different from the at least one fixing polymer; and
(iii) at least one antidandruff agent.

17. The cosmetic composition according to claim 16, wherein the at least one fixing polymer is chosen from anionic fixing polymers, cationic fixing polymers, amphoteric fixing polymers, and nonionic fixing polymers.

18. The cosmetic composition according to claim 17, wherein the at least one fixing polymer is a cationic fixing polymer chosen from homopolymers or copolymers of acrylic or methacrylic esters or amides containing amine functions, cationic guar gums, quaternary copolymers of vinylpyrrolidone and of vinylimidazole, and chitosans.

19. The cosmetic composition according to claim 17, wherein the at least one fixing polymer is an anionic fixing polymer chosen from copolymers of acrylic acid or methacrylic acid or salts thereof, crotonic acid copolymers, copolymers of carboxylic acids or anhydrides, polyacrylamides containing carboxylate groups, homopolymers or copolymers containing sulfonic groups, anionic polyurethanes, and anionic grafted silicone polymers.

20. The cosmetic composition according to claim 17, wherein the at least one fixing polymers is an amphoteric fixing polymer chosen from copolymers containing acidic vinyl units and basic vinyl units, crosslinked and acylated polyaminoamides, polymers containing zwitterionic units, chitosan-based polymers, and modified (C₁-C₅)alkyl vinyl ether/maleic anhydride copolymers.

21. The cosmetic composition according to claim 17, wherein the at least one fixing polymer is a nonionic fixing polymer chosen from:
vinylpyrrolidone homopolymers;
polyalkyloxazolines;
vinyl acetate homopolymers;
vinyl acetate copolymers;
acrylic ester homopolymers and copolymers;
copolymers of acrylonitrile and of a nonionic monomer;
styrene homopolymers;
styrene copolymers;
polyamides;
vinyllactam homopolymers; and
vinylpyrrolidone homopolymers.

22. The cosmetic composition according to claim 21, wherein the nonionic fixing polymer is polyvinylcaprolactam.

23. The cosmetic composition according to claim 16, wherein the concentration of the at least one fixing polymer ranges from about 0.1% to about 10% by weight, relative to the total weight of the composition.

24. The cosmetic composition according to claim 16, wherein the concentration of the at least one fixing polymer ranges from about 0.5% to about 5% by weight, relative to the total weight of the composition.

25. The cosmetic composition according to claim 16, wherein the at least one associative polymer is chosen from anionic associative polymers, nonionic associative polymers, cationic associative polymers, and amphoteric associative polymers.

26. The cosmetic composition according to claim 25, wherein the at least one associative polymer is chosen from anionic associative polymers containing at least one unsaturated olefinic carboxylic acid hydrophilic unit and at least one unsaturated (C₁₀-C₃₀)alkylcarboxylic acid ester hydrophobic unit.

27. The cosmetic composition according to claim 16, wherein the concentration of the at least one associative polymer ranges from about 0.1% to about 10% by weight, relative to the total weight of the composition.

28. The cosmetic composition according to claim 16, wherein the concentration of the at least one associative polymer ranges from about 0.5% to about 5% by weight, relative to the total weight of the composition.

29. The cosmetic composition according to claim 16, wherein the at least one antidandruff agent is chosen from:
1) pyridinethione salts,
2) 1-hydroxy-2-pyrrolidone derivatives,
3) 2,2′-dithiobis(pyridine N-oxide),
4) trihalocarbamides,
5) triclosan,
6) azole compounds,
7) selenium sulfides,
8) extracts of at least one non-photosynthetic, non-fruiting filamentous bacteria,
9) antifungal polymers, and
10) ulphid in its various forms, cadmium ulphide, allantoin, coal or wood tars and derivatives thereof, salicylic acid, undecylenic acid, fumaric acid, ellagic acid, ellagic acid tannins, and allylamines.

30. The cosmetic composition according to claim 29, wherein the at least one antidandruff agent is an azole compound chosen from climbazole, ketoconazole, clotrimazole, econazole, isoconazole and miconazole.

31. The cosmetic composition according to claim 29, wherein the at least one antidandruff agent is an antifungal polymer chosen from amphotericin B or nystatin.

32. The cosmetic composition according to claim 16, wherein the at least one antidandruff agent is chosen from 1-hydroxy-4-methyl-6-(2,4,4-trimethylpentyl)-2-pyridone and salts thereof.

33. The cosmetic composition according to claim 16, wherein the concentration of the at least one antidandruff agent ranges from about 0.001% to about 10% by weight, relative to the total weight of the composition.

34. The cosmetic composition according to claim 16, wherein the concentration of the at least one antidandruff agent ranges from about 0.2% to about 2% by weight, relative to the total weight of the composition.

35. A process for treating keratin materials comprising applying to the keratin materials a cosmetic composition comprising, in a cosmetically acceptable medium:
(i) at least one fixing polymer;
(ii) at least one associative polymer different from the at least one fixing polymer; and
(iii) at least one antidandruff agent.
